(19) United States
(12) Patent Application Publication
(10) Pub. No.: US 2026/0051367 A1
(43) Pub. Date: Feb. 19, 2026

(54) AUTOMATED CIRCUIT OPTIMIZATION FOR QUANTUM CHEMISTRY

(71) Applicant: IonQ, Inc., College Park, MD (US)

(72) Inventors: Andrii MAKSYMOV, Hyattsville, MD (US); Felix Tripier, Washington, DC (US)

(21) Appl. No.: 19/300,210

(22) Filed: Aug. 14, 2025

Related U.S. Application Data
(60) Provisional application No. 63/684,090, filed on Aug. 16, 2024.

(51) Int. Cl. G16C 10/00; G06N 10/60

(52) U.S. Cl. CPC G16C 10/00; G06N 10/60

(57) ABSTRACT

Aspects of the present disclosure relate generally to systems and methods for optimizing an implementation of a quantum chemistry task in a quantum circuit. The method includes obtaining a chemistry task presented as a Hamilton to implement in a Variational Quantum Eigensolver (VQE) circuit. The method also includes mapping the chemistry task to a qubit space of the VQE circuit with Unitary Coupled Cluster with Single and Double excitations (UCCSD) ansatz. The method also includes decomposing the fermionic excitations to Givens rotations and controlled-Z (CZ) gates by passing exponentiated Pauli forms in the VQE circuit to a cloud compiler. The method further includes after commuting the CZ gates through Givens rotations, cancelling the commuted CZ gates. The method further includes dropping CZ gates at the beginning and the end of the quantum circuit.

### CROSS REFERENCE TO RELATED APPLICATIONS

This application claims priority to U.S. Patent Provisional Application No. 63/684,090, filed Aug. 16, 2024, the contents of which are hereby incorporated by reference in its entirety.

### TECHNICAL FIELD

Aspects of the present disclosure relate generally to systems and methods for use in the implementation, operation, and/or use of optimizing implementations of quantum chemistry tasks on quantum circuits.

### BACKGROUND

Variational Quantum Eigensolver (VQE) is a quantum-based method designed for finding a minimum eigenvalue of a given Hamiltonian, which describes the energy of a quantum system. The VQE works by parameterizing a trial wavefunction encoded as a quantum circuit. Parameters in that trial wavefunction are optimized by iteratively running an encoding circuit and using a classical optimization algorithm to vary the parameters in order to minimize the expectation of the Hamilton. This optimization process may be repeated until a minimum eigenvalue is found to be a satisfactory degree of accuracy.

### SUMMARY

The following presents a simplified summary of one or more aspects to provide a basic understanding of such aspects. This summary is not an extensive overview of all contemplated aspects and is intended to neither identify key or critical elements of all aspects nor delineate the scope of any or all aspects. Its sole purpose is to present some concepts of one or more aspects in a simplified form as a prelude to the more detailed description that is presented later.

This disclosure describes various aspects of techniques for compiling and implementing quantum chemistry tasks on VQE quantum circuits. Specifically, the present disclosure describes preserving the original high-level structure of VQE circuits in terms of exponential Pauli operators and Givens rotations. Doing so allows for more efficient circuit optimization and error mitigation. This is in contrast to related embodiments where higher-level functional blocks are quickly lowered to a reduced gate-set. Preserving high-level expressions of the problem better enable optimizations that reduce circuit depth and improve fidelity for various chemistry tasks.

In some aspects of the present disclosure, a method for optimizing an implementation of a quantum chemistry task in a quantum circuit is described. The method includes: obtaining a chemistry task presented as a Hamilton to implement in a Variational Quantum Eigensolver (VQE) circuit; mapping the chemistry task to a qubit space of the VQE circuit with Unitary Coupled Cluster with Single and Double excitations (UCCSD) ansatz comprising a state preparation, single and double fermionic excitations, and a measurement basis, wherein fermionic excitation operators in the qubit space are implemented as Givens rotations dressed with controlled-Z (CZ) gate ladders at a beginning and end of the quantum circuit; decomposing the fermionic excitations to Givens rotations and controlled-Z (CZ) gates by passing exponentiated Pauli forms in the VQE circuit to a cloud compiler; after commuting the CZ gates through Givens rotations, cancelling the commuted CZ gates; and dropping CZ gates at the beginning and the end of the quantum circuit.

In some aspects of this present disclosure, a system configured to optimize an implementation of a quantum chemistry task in a quantum circuit is described. The system includes at least one processor and a memory including instructions that, when executed by the at least one processor, cause the system to: obtain a chemistry task presented as a Hamilton to implement in a Variational Quantum Eigensolver (VQE) circuit; map the chemistry task to a qubit space of the VQE circuit with Unitary Coupled Cluster with Single and Double excitations (UCCSD) ansatz comprising a state preparation, single and double fermionic excitations, and a measurement basis, wherein fermionic excitation operators in the qubit space are implemented as Givens rotations dressed with controlled-Z (CZ) gate ladders at a beginning and end of the VQE circuit; decompose the fermionic excitations to Givens rotations and controlled-Z (CZ) gates by passing exponentiated Pauli forms in the VQE circuit to a cloud compiler; after commuting the CZ gates through Givens rotations, cancel the commuted CZ gates; and drop CZ gates at the beginning and the end of the VQE circuit.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

The disclosed aspects will hereinafter be described in conjunction with the appended drawings, provided to illustrate and not to limit the disclosed aspects, wherein like designations denote like elements, and in which:

FIG. 1 illustrates a view of atomic ions in a linear crystal or chain in accordance with aspects of this disclosure.

FIG. 2 illustrates an example of a quantum information processing (QIP) system in accordance with aspects of this disclosure.

FIG. 3 illustrates an example of a computer device in accordance with aspects of this disclosure.

FIG. 4 illustrates a flowchart of an example of implementing VQE as an application in accordance with aspects of this disclosure.

FIG. 5 illustrates an example of an optimization pass optimizing the decomposition of fermionic excitations to reduce gate count in a quantum circuit in accordance with aspects of this disclosure.

FIG. 6 illustrates an example of a four-qubit Givens rotation decomposition in accordance with aspects of this disclosure.

FIG. 7 illustrates a method for optimizing an implementation of a quantum chemistry task in a quantum circuit in accordance with aspects of this disclosure.

FIG. 8 illustrates an example of a QIP system in accordance with aspects of this disclosure.

Like reference numbers and designations in the various drawings indicate like elements.

### DETAILED DESCRIPTION

The detailed description set forth below in connection with the appended drawings or figures is intended as a description of various configurations or implementations and is not intended to represent the only configurations or implementations in which the concepts described herein may be practiced. The detailed description includes specific details for the purpose of providing a thorough understanding of various concepts. However, it will be apparent to those skilled in the art that these concepts may be practiced without these specific details or with variations of these specific details. In some instances, well known components are shown in block diagram form, while some blocks may be representative of one or more well-known components.

The exemplary system and method herein provides for optimization of implementing a chemistry task in a VQE circuit. In particular, the system and method includes adding compiler support for high-level circuit primitives such as fermionic excitations through exponentiated Pauli sums and Givens rotations. The present disclosure preserves the original high-level structure of VQE circuits in terms of exponentiated Pauli operators and Givens rotations. Doing so allows for more efficient circuit optimization and error mitigation. This is in contrast to a more common paradigm where higher-level functional blocks are quickly lowered to a reduced gateset. In addition, preserving high-level expressions of the problem better enable optimizations that reduce depth and improve fidelity for various chemistry tasks including a Unitary Coupled Cluster with Single and Double excitations (UCCSD) ansatz from a repository.

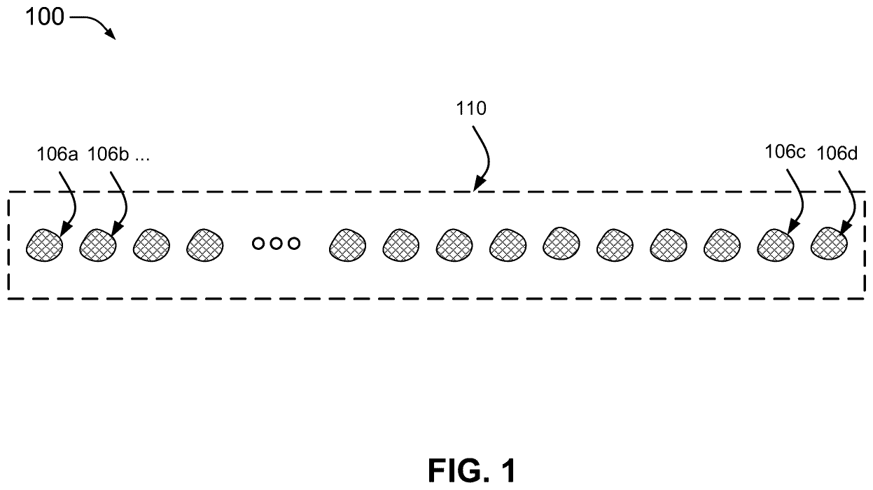

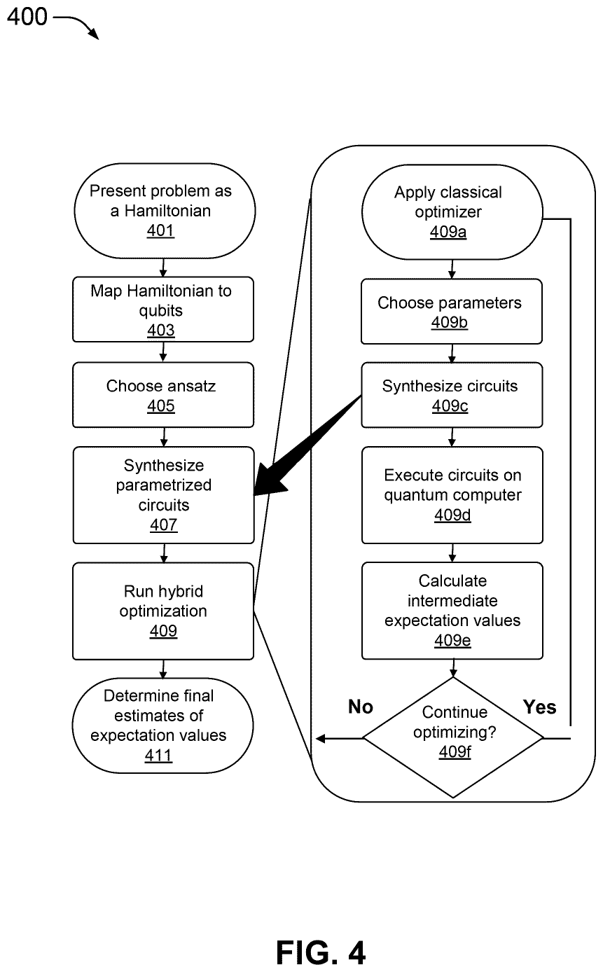

Solutions to the issues described above are explained in more detail in connection with FIGS. 1-8, with FIGS. 1-3 and 8 providing a general disclosure of QIP systems or quantum computers, and more specifically, of atomic based QIP systems or quantum computers, FIGS. 4-8 provide descriptions and examples of optimizing an implementation of a quantum chemistry task in a quantum circuit, in accordance with various example aspects of the present disclosure.

Trapped atoms are one of the leading implementations for quantum information processing or quantum computing. Atomic-based qubits may be used as quantum memories, as quantum gates in quantum computers and simulators, and may act as nodes for quantum communication networks. Qubits based on trapped atomic ions enjoy a rare combination of attributes. For example, qubits based on trapped atomic ions have very good coherence properties, may be prepared and measured with nearly 100% efficiency, and are readily entangled with each other by modulating their Coulomb interaction with suitable external control fields such as optical or microwave fields. These attributes make atomic-based qubits attractive for extended quantum operations such as quantum computations or quantum simulations.

Atomic quantum computers can include array(s) of atoms or ions trapped, for example, inside a vacuum chamber. A size and dimensionality of atomic arrays may vary.

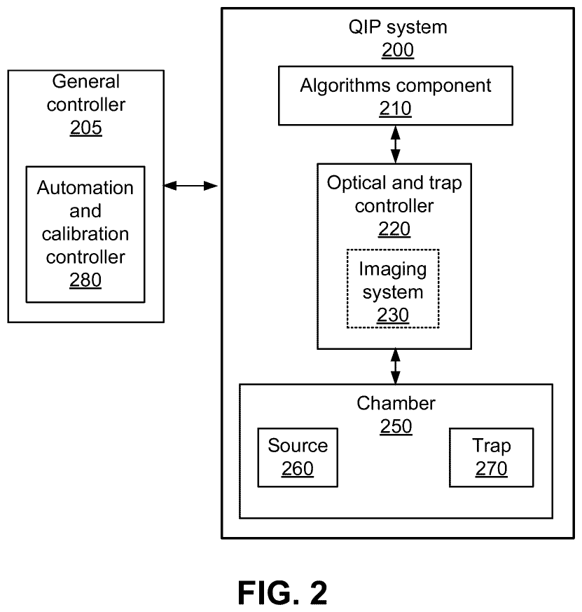

FIG. 1 illustrates a diagram 100 with multiple atomic ions or ions 106 (e.g., ions 106a, 106b, . . . , 106c, and 106d) trapped in a linear crystal or chain 110 using a trap (not shown; the trap can be inside a vacuum chamber as shown in FIG. 2). The trap maybe referred to as an ion trap. The ion trap shown may be built or fabricated on a semiconductor substrate, a dielectric substrate, or a glass die or wafer (also referred to as a glass substrate). The ions 106 may be provided to the trap as atomic species for ionization and confinement into the chain 110. Some or all of the ions 106 may be configured to operate as qubits in a QIP system.

In the example shown in FIG. 1, the trap includes electrodes for trapping or confining multiple ions into the chain 110 laser-cooled to be nearly at rest. The number of ions trapped can be configurable and more or fewer ions may be trapped. The ions can be ytterbium ions (e.g., ¹⁷¹Yb⁺ ions), for example. The ions are illuminated with laser (optical) radiation tuned to a resonance in ¹⁷¹Yb⁺ and the fluorescence of the ions is imaged onto a camera or some other type of detection device (e.g., photomultiplier tube or PMT). In this example, ions may be separated by a few microns (μm) from each other, although the separation may vary based on architectural configuration. The separation of the ions is determined by a balance between the external confinement force and Coulomb repulsion and does not need to be uniform. Moreover, in addition to ytterbium ions, barium ions, neutral atoms, Rydberg atoms, or other types of atomic-based qubit technologies may also be used. Moreover, ions of the same species, ions of different species, and/or different isotopes of ions may be used. The trap may be a linear RF Paul trap, but other types of confinement devices may also be used, including optical confinements. Thus, a confinement device may be based on different techniques and may hold ions, neutral atoms, or Rydberg atoms, for example, with an ion trap being one example of such a confinement device. The ion trap may be a surface trap, for example.

The chain 110 of ions 106 may be part of a QPU, that is, the chain 110 of ions 106 may be part of a processing engine or processing core of a QIP system. When any one of the ions 106 is capable of being connected to any other ion 106 in the chain 110, the chain 110 is considered to be fully connected, and thus, it can be used to implement a fully connected QPU. Fully connected QPUs need not be limited to atomic-based QIP systems.

FIG. 2 illustrates a block diagram that shows an example of a QIP system 200. The QIP system 200 may also be referred to as a quantum computing system, a quantum computer, a computer device, a trapped ion system, or the like. The QIP system 200 may be part of a hybrid computing system in which the QIP system 200 is used to perform quantum computations and operations and the hybrid computing system also includes a classical computer to perform classical computations and operations. The quantum and classical computations and operations may interact in such a hybrid system.

Shown in FIG. 2 is a general controller 205 configured to perform various control operations of the QIP system 200. These control operations may be performed by an operator, may be automated, or a combination of both. Instructions for at least some of the control operations may be stored in memory (not shown) in the general controller 205 and may be updated over time through a communications interface (not shown). Although the general controller 205 is shown separate from the QIP system 200, the general controller 205 may be integrated with or be part of the QIP system 200. The general controller 205 may include an automation and calibration controller 280 configured to perform various calibration, testing, and automation operations associated with the QIP system 200. These calibration, testing, and automation operations may involve, for example, all or part of an algorithms component 210, all or part of an optical and trap controller 220 and/or all or part of a chamber 250.

The QIP system 200 may include the algorithms component 210 mentioned above, which may operate with other parts of the QIP system 200 to perform or implement quantum algorithms, quantum applications, or quantum operations. The algorithms component 210 may be used to perform or implement a stack or sequence of combinations of single qubit operations and/or multi-qubit operations (e.g., two-qubit operations) as well as extended quantum computations. The algorithms component 210 may also include software tools (e.g., compilers) that facility such performance or implementation. As such, the algorithms component 210 may provide, directly or indirectly, instructions to various components of the QIP system 200 (e.g., to the optical and trap controller 220) to enable the performance or implementation of the quantum algorithms, quantum applications, or quantum operations. The algorithms component 210 may receive information resulting from the performance or implementation of the quantum algorithms, quantum applications, or quantum operations and may process the information and/or transfer the information to another component of the QIP system 200 or to another device (e.g., an external device connected to the QIP system 200) for further processing.

The QIP system 200 may include the optical and trap controller 220 mentioned above, which controls various aspects of a trap 270 in the chamber 250, including the generation of signals to control the trap 270. The optical and trap controller 220 may also control the operation of lasers, optical systems, and optical components that are used to provide the optical beams that interact with the atoms or ions in the trap. Optical systems that include multiple components may be referred to as optical assemblies. The optical beams are used to set up the ions, to perform or implement quantum algorithms, quantum applications, or quantum operations with the ions, and to read results from the ions. Control of the operations of laser, optical systems, and optical components may include dynamically changing operational parameters and/or configurations, including controlling positioning using motorized mounts or holders. When used to confine or trap ions, the trap 270 may be referred to as an ion trap. The trap 270, however, may also be used to trap neutral atoms, Rydberg atoms, and other types of atomic-based qubits. The lasers, optical systems, and optical components can be at least partially located in the optical and trap controller 220, an imaging system 230, and/or in the chamber 250.

The QIP system 200 may include the imaging system 230. The imaging system 230 may include a high-resolution imager (e.g., CCD camera) or other type of detection device (e.g., PMT) for monitoring the ions while they are being provided to the trap 270 and/or after they have been provided to the trap 270 (e.g., to read results). In an aspect, the imaging system 230 can be implemented separate from the optical and trap controller 220, however, the use of fluorescence to detect, identify, and label ions using image processing algorithms may need to be coordinated with the optical and trap controller 220.

In addition to the components described above, the QIP system 200 can include a source 260 that provides atomic species (e.g., a plume or flux of neutral atoms) to the chamber 250 having the trap 270. When atomic ions are the basis of the quantum operations, that trap 270 confines the atomic species once ionized (e.g., photoionized). The trap 270 may be part of what may be referred to as a processor or processing portion of the QIP system 200. That is, the trap 270 may be considered at the core of the processing operations of the QIP system 200 since it holds the atomic-based qubits that are used to perform or implement the quantum operations or simulations. At least a portion of the source 260 may be implemented separate from the chamber 250.

It is to be understood that the various components of the QIP system 200 described in FIG. 2 are described at a high-level for ease of understanding. Such components may include one or more sub-components, the details of which may be provided below as needed to better understand certain aspects of this disclosure.

Aspects of this disclosure may be implemented at least partially using one or more of the general controller 205, the automation and calibration controller 280, the optical and trap controller 220, and the chamber 250.

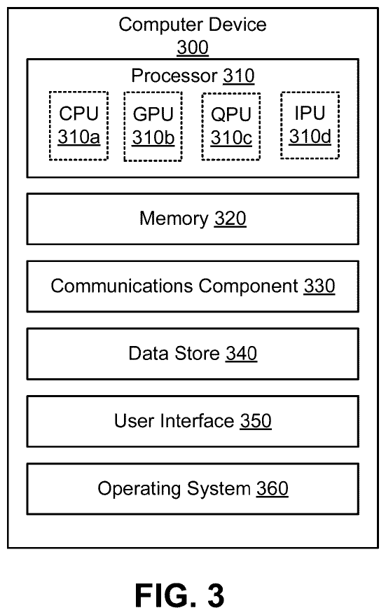

Referring now to FIG. 3, an example of a computer system or device 300 is shown. The computer device 300 may represent a single computing device, multiple computing devices, or a distributed computing system, for example. The computer device 300 may be configured as a quantum computer (e.g., a QIP system), a classical computer, or to perform a combination of quantum and classical computing functions, sometimes referred to as hybrid functions or operations. For example, the computer device 300 may be used to process information using quantum algorithms, classical computer data processing operations, or a combination of both. In some instances, results from one set of operations (e.g., quantum algorithms) are shared with another set of operations (e.g., classical computer data processing). A generic example of the computer device 300 implemented as a QIP system capable of performing quantum computations and simulations is, for example, the QIP system 200 shown in FIG. 2.

The computer device 300 may include a processor 310 for carrying out processing functions associated with one or more of the features described herein. The processor 310 may include a single processor, multiple set of processors, or one or more multi-core processors. Moreover, the processor 310 may be implemented as an integrated processing system and/or a distributed processing system. The processor 310 may include one or more central processing units (CPUs) 310a, one or more graphics processing units (GPUs) 310b, one or more quantum processing units (QPUs) 310c, one or more intelligence processing units (IPUs) 310d (e.g., artificial intelligence or AI processors), or a combination of some or all those types of processors. In one aspect, the processor 310 may refer to a general processor of the computer device 300, which may also include additional processors 310 to perform more specific functions (e.g., including functions to control the operation of the computer device 300). Quantum operations may be performed by the QPUs 310c. Some or all of the QPUs 310c may use atomic-based qubits, however, it is possible that different QPUs are based on different qubit technologies. One or more of the QPUs 310c may be fully connected QPUs in accordance with aspects of this disclosure.

The computer device 300 may include a memory 320 for storing instructions executable by the processor 310 to carry out operations. The memory 320 may also store data for processing by the processor 310 and/or data resulting from processing by the processor 310. In an implementation, for example, the memory 320 may correspond to a computer-readable storage medium that stores code or instructions to perform one or more functions or operations. Just like the processor 310, the memory 320 may refer to a general memory of the computer device 300, which may also include additional memories 320 to store instructions and/or data for more specific functions.

It is to be understood that the processor 310 and the memory 320 may be used in connection with different operations including but not limited to computations, calculations, simulations, controls, calibrations, system management, and other operations of the computer device 300, including any methods or processes described herein.

Further, the computer device 300 may include a communications component 330 that provides for establishing and maintaining communications with one or more parties utilizing hardware, software, and services. The communications component 330 may also be used to carry communications between components on the computer device 300, as well as between the computer device 300 and external devices, such as devices located across a communications network and/or devices serially or locally connected to computer device 300. For example, the communications component 330 may include one or more buses, and may further include transmit chain components and receive chain components associated with a transmitter and receiver, respectively, operable for interfacing with external devices. The communications component 330 may be used to receive updated information for the operation or functionality of the computer device 300.

Additionally, the computer device 300 may include a data store 340, which can be any suitable combination of hardware and/or software, which provides for mass storage of information, databases, and programs employed in connection with the operation of the computer device 300 and/or any methods or processes described herein. For example, the data store 340 may be a data repository for operating system 360 (e.g., classical OS, or quantum OS, or both). In one implementation, the data store 340 may include the memory 320. In an implementation, the processor 310 may execute the operating system 360 and/or applications or programs, and the memory 320 or the data store 340 may store them.

The computer device 300 may also include a user interface component 350 configured to receive inputs from a user of the computer device 300 and further configured to generate outputs for presentation to the user or to provide to a different system (directly or indirectly). The user interface component 350 may include one or more input devices, including but not limited to a keyboard, a number pad, a mouse, a touch-sensitive display, a digitizer, a navigation key, a function key, a microphone, a voice recognition component, any other mechanism capable of receiving an input from a user, or any combination thereof. Further, the user interface component 350 may include one or more output devices, including but not limited to a display, a speaker, a haptic feedback mechanism, a printer, any other mechanism capable of presenting an output to a user, or any combination thereof. In an implementation, the user interface component 350 may transmit and/or receive messages corresponding to the operation of the operating system 360. When the computer device 300 is implemented as part of a cloud-based infrastructure solution, the user interface component 350 may be used to allow a user of the cloud-based infrastructure solution to remotely interact with the computer device 300.

Thus, the systems and methods described herein are configured to optimize an implementation of a quantum chemistry task in a quantum circuit. For example, a native gate set is a set of quantum gates that can be physically executed on hardware computing systems (e.g., FIGS. 2-3) by addressing ions (e.g., the exemplary ion chain in FIG. 1) with resonant lasers via stimulated Raman transitions. The angle θ can be defined by the amount of X-rotation where single-qubit gates can be rotated along different axes on a Bloch sphere and/or as rotations along a fixed axis while rotating the Bloch sphere itself. In an exemplary aspect, the rotations can be physically implemented as Rabi oscillations that are made with a two-photon Raman transition to drive the plurality of qubits, such as the ion chain shown in FIG. 1, for example, on resonance using a pair of lasers in a Raman configuration that can be implemented by the optical and trap controller 220, for example. Moreover, the ranges can be controlled by varying the duration of the laser pulses of the Raman configuration.

In connection with the systems described in FIGS. 1-3 and 8, a technique or method for optimizing an implementation of a quantum chemistry task in a quantum circuit is described. Specifically, the present disclosure describes a method of adding compiler support for high-level circuit primitives such as fermionic excitations through exponentiated Pauli sums and Givens rotations. In addition, the present disclosure describes a workflow whereby the exponentiated Pauli forms in VQE circuits are passed directly to the cloud compiler such that the relevant optimization passes can operate directly on that richer representation to decompose them down to native gates more efficiently. The systems described in FIGS. 2 and/or 3 may be used to control various aspects of the QIP system as described below.

FIG. 4 illustrates a flowchart of an example of implementing VQE as an application in accordance with aspects of this disclosure. Method 400 of FIG. 4 shows a typical model pipeline using VQE as an application. VQE is a hybrid variational algorithm used to find various properties related to the ground state of a quantum system.

The main objective of the pipeline is to create a circuit that generates a good approximation of the target expectation value such as ground state of an input Hamiltonian. At each optimization step, new circuits are generated and optimized. If the circuits are given in terms of high-level primitives than most of the circuit optimization work may be done outside of the parameter optimization loop, reducing classical overheads.

At step 401, the method 400 includes presenting a problem as a Hamiltonian.

At step 403, the method 400 includes mapping the Hamiltonian to a qubit space.

At step 405, the method 400 includes choosing ansatz. Ansatz are a parameterized quantum circuit meant to produce a good approximation of the desired ground state if the right parameters are chosen.

At step 407, the method 400 includes synthesizing parameterized circuits.

At step 409, the method 400 includes running a hybrid optimization using variational methods. This is an iterative process 409 which requires running the circuit with given parameters and then using the result to update those parameters with the aid of a classical optimizer. This optimization is repeated many times in order to gain a good approximation of the target observable such as ground state energy.

Specifically, the iterative process 409 includes, at step 409a, the iterative process 409 includes applying a classical optimizer. At step 409b, the iterative process 409 includes choosing parameters. At step 409c, the iterative process 409 includes synthesizing circuits. At step 409d, the iterative process 409 includes executing circuits on quantum computer. At step 409e, the iterative process 409 includes calculating intermediate expectation values. At step 409f, the iterative process 409 includes determining whether to continue optimization. If the decision is to continue optimization, then the iterative process 409 continues back to applying the classical optimizer at step 409a. If the decision is to not continue optimization, then the iterative process 409 ends and the method 400 continues to step 411.

At step 411, the method 400 includes determining final estimates of expectation values.

There are many way to optimize circuit synthesis for quantum chemistry and VQE by using the symmetries of the target Hamiltonian. Assuming the symmetries of the Hamiltonian are unknown at the compiler level, the present disclosure describes an approach to circuit optimization that relies only on the symmetries of the circuit itself and a set of local circuit identifies defined for an extended gate set.

The extended gateset may be comprised of exponentiated Pauli operations and Givens rotations, which are common in quantum chemistry. Givens rotations are particle-number conserving operations that implement an R_{y} rotation between half-populated states (e.g., between 01 and 10 states for two qubits and between 0101 and 1010 states for four qubits). The present disclosure also proposes a workflow whereby the exponentiated optimization passes can operate directly on that richer representation in order to decompose them down to native gates much more efficiently.

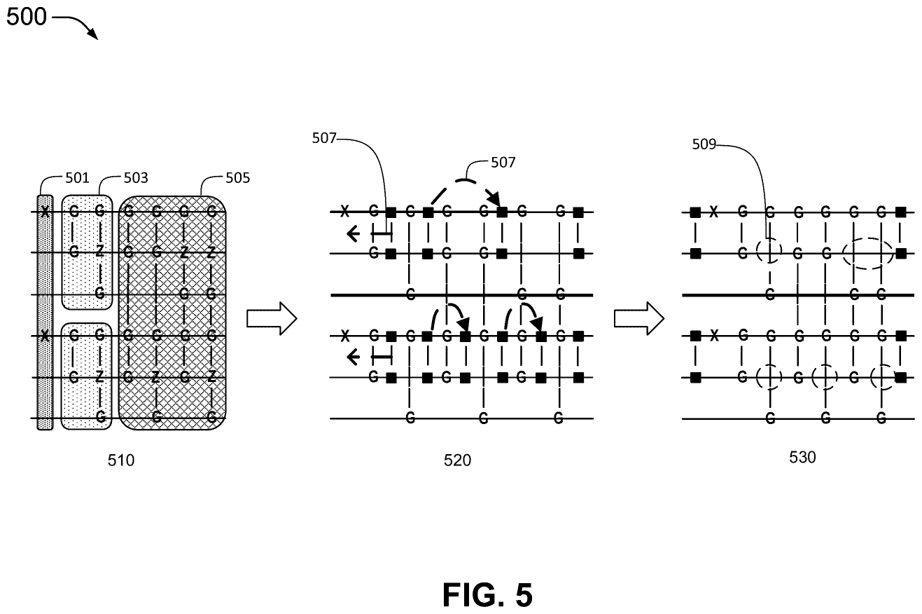

FIG. 5 illustrates an example of an optimization pass optimizing the decomposition of fermionic excitations operators to reduce gate count in a quantum circuit in accordance with aspects of this disclosure. Fermionic excitation operators in qubit space can be implemented as Givens rotations dressed with Controlled-Z (CZ) gate ladders on both sides to realize πᵢZᵢ terms. There is a freedom in choosing the placement of these CZ gates. That freedom can be used alongside its commutation properties with respect to Givens rotations (marked with G on the circuits) to cancel out many CZ operators (marked with Z on the circuits).

The example 500 in FIG. 5 describes an optimization pass optimizing the decomposition of fermionic excitations for reducing 2q-gate count. In some examples, the decomposition of fermionic excitations reduces 2q-gate count from 89 to 50 for a VQE circuit.

At 510, example 500 shows a VQE circuit with a UCCSD ansatz consists of state preparation 501 (e.g., 1q-qubit X gates), single 503 and double fermionic excitations 505, and a measurement basis. The measurement basis has no additional gates here because of the Z basis.

At 520, example 500 shows decomposing 507 the fermionic excitations to Givens rotations and CZ gates.

At 530, example 500 shows, after commuting CZ gates through Givens rotations (where possible) and canceling the CZ gates out 509 (depicted as dotted circles), the remaining CZ gates at the end and beginning of the circuit can be dropped.

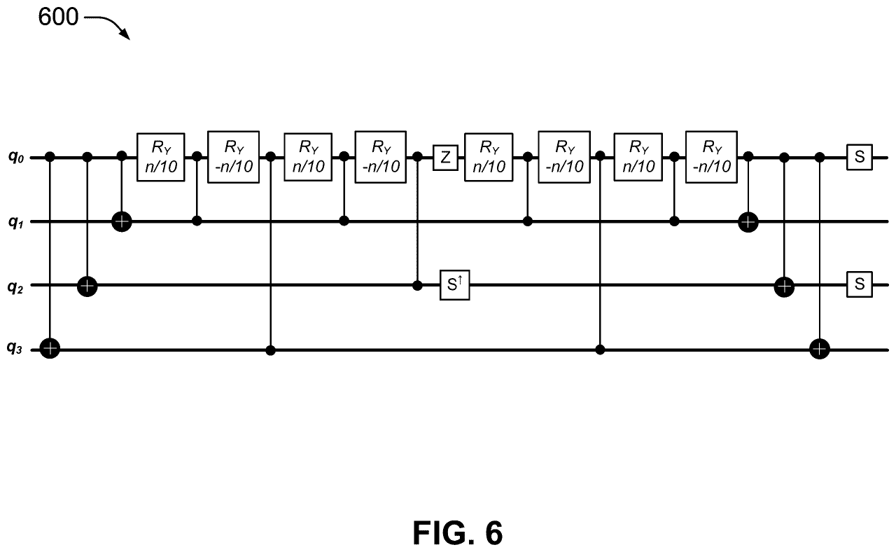

FIG. 6 illustrates an example 600 of a four-qubit Givens rotation decomposition in accordance with aspects of this disclosure. Example 600 of FIG. 6 shows a four-qubit Givens rotation decomposition to 13 2q gates in the form that reduces beam reassignment compared to a default ladder decompositions of exponentiated Paulis.

The remaining Givens rotations can be decomposed with only 2q gates for single excitations and 13 2q gates for double excitations. Due to the order of double excitations, alternating polarity of decompositions, additional 2q gates that comprise neighboring 4q Givens rotations can be cancelled. As an example, for VQE06 that leads to 60 2q gates after canceling out CZ gates and up to 50 2q gates after canceling out CX gates between 4q Givens rotations.

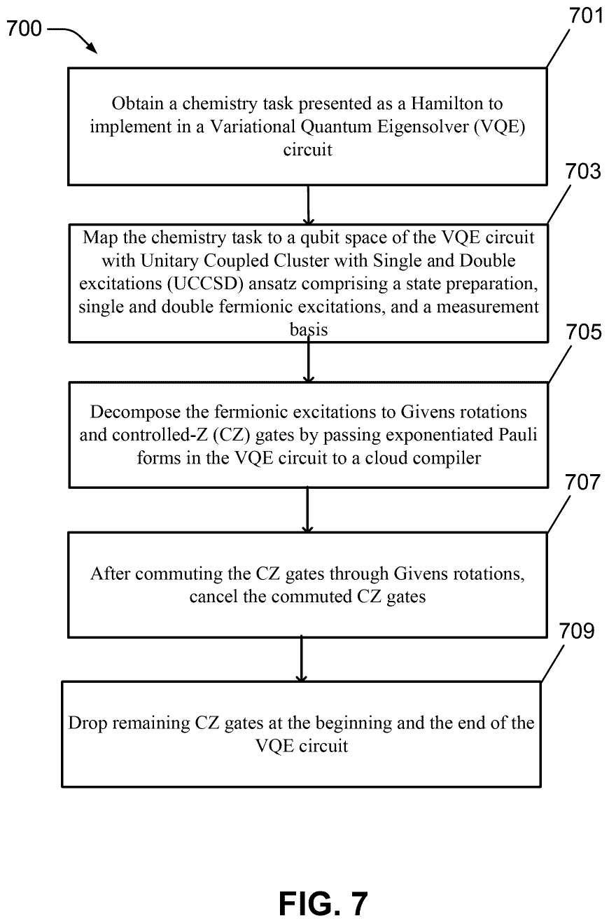

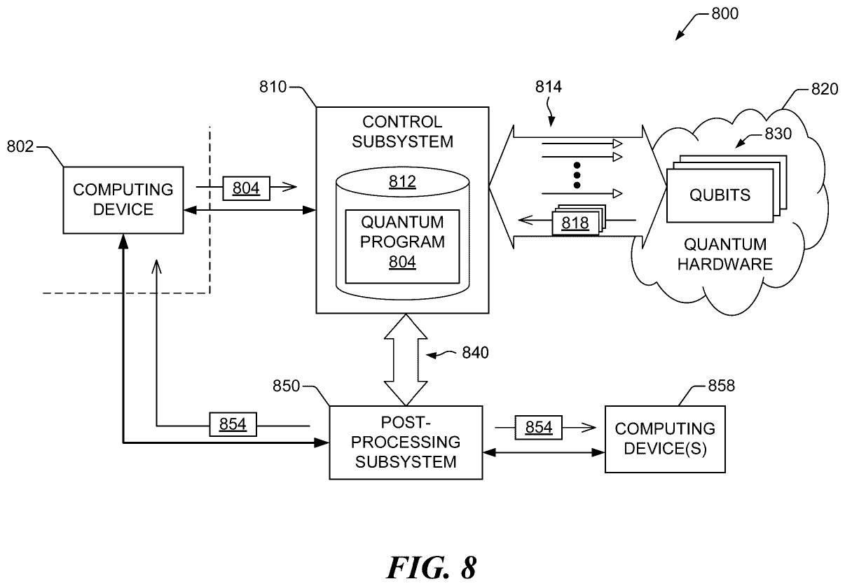

FIG. 7 illustrates a method for optimizing an implementation of a quantum chemistry task in a quantum circuit in accordance with aspects of this disclosure. In general, it is noted that parts of the exemplary method 700 can be implemented using the components and systems described herein, especially with request to QIP system 200 and general controller 205 of FIG. 2 as described above and QIP system 800 of FIG. 8 described below. The steps and algorithms described in relation to method 700 may be executed by processor 310 using algorithms components 210. Specifically, the method 700 in FIG. 7 describes an approach to circuit optimization that relies only on the symmetries of the VQE circuit itself and a set of local circuit identities defined for an extended gateset.

At step 701, the method 700 may include obtaining a chemistry task presented as a Hamilton to implement in a Variational Quantum Eigensolver (VQE) circuit.

At step 703, the method 700 may include mapping the chemistry task to a qubit space of the VQE circuit with Unitary Coupled Cluster with Single and Double excitations (UCCSD) ansatz comprising a state preparation, single and double fermionic excitations, and a measurement basis. The fermionic excitation operators in the qubit space may be implemented as Givens rotations dressed with controlled-Z (CZ) gate ladders at a beginning and end of the VQE circuit. The state preparation may include a 1q-qubit X gates.

At step 705, the method 700 may include decomposing the fermionic excitations to Givens rotations and controlled-Z (CZ) gates by passing exponentiated Pauli forms in the VQE circuit to a cloud compiler.

At step 707, the method 700 may include after commuting the CZ gates through Givens rotations, cancelling the commuted CZ gates.

At step 709, the method 700 may include dropping CZ gates at the beginning and the end of the VQE circuit.

Optionally, the method 700 may further include executing the VQE circuit having the dropped CZ gates for reducing gate counts in the VQE circuit. The updated VQE circuit is executed by encoding a quantum state using high-level primitives such as exponentiated Pauli operators and Givens rotations. These are then efficiently decomposed into a lower-level gateset through compiler optimization passes. The resulting circuit is run iteratively on quantum hardware, with parameters updated by a classical optimizer to minimize the Hamiltonian's expectation value. Preserving the high-level structure enables optimizations that exploit commutation and symmetry, reducing two-qubit gates, circuit depth, and improving fidelity.

Optionally, the method 700 may further include decomposing remaining Givens rotations with two 2q gates for single excitations and thirteen 2q gates for double excitations. In some examples, the method 700 may further include alternating polarity of decomposition to cancel an additional 2q gates comprising neighboring 4q Givens rotations.

The disclosure provides a technique for optimizing an implementation of a quantum chemistry task in a quantum circuit. By representing application circuits in terms of higher-level primitives, the present disclosure helps with error mitigation, designing non-local twirling patterns and dynamical decoupling strategies since it is easier when large block structures (such as 4q Givens rotation subcircuits) are apparent. Symmetries and commutation properties of such primitives can also be used to better automate the search for error detection gadgets and constraints on output bit-strings without knowing implicit symmetries of the modeled system.

It is understood that the method illustrated by FIG. 7 is exemplary in nature and that the steps described herein may be combined or modified to generate alternative embodiments.

FIG. 8 illustrates an example of a QIP system in accordance with aspects of this disclosure. The example QIP system 800 shown in FIG. 8 includes a control subsystem 810 that can receive a quantum program 804 from a computing device 802 that is remotely located relative to the example QIP system 800 and is functionally coupled (e.g., communicatively coupled) to the control subsystem 810. The computing device 802 can send data defining the quantum program 804 to control subsystem 810 for execution, in quantum hardware 820, to optimize an implementation of a quantum chemistry task in a quantum circuit, as described herein. As is indicated by dashed lines, the computing device 802 can be external to the example QIP system 800. For example, the computing device 802 can be a user device (e.g., a classical computer) of an end-user of the QIP system 800. The control subsystem 810 can retain the quantum program 804 in one or more memory devices 812. The quantum program 804 corresponds to a defined quantum computation. The defined quantum computation can be an n-qubit computation, for example. The quantum program 804 can include a quantum circuit (and, in some cases, sub-circuits, such as the mapping component, decomposition component, and configuration component) representing a quantum algorithm associated with the quantum computation. Examples of the quantum algorithm include a variational quantum algorithm, a machine-learning algorithm, a Fourier transform algorithm, or the like.

The control subsystem 810 can be functionally coupled to quantum hardware 820 via multiple links 814 that permits the exchange of data and/or controls signal between the control subsystem 810 and the quantum hardware 820. The quantum hardware 820 can embody or can include one or more quantum computers. In some cases, the quantum hardware 820 embodies a cloud-based quantum computer. In other cases, the quantum hardware 820 embodies, or includes a local quantum computer. Regardless of its spatial footprint, the quantum hardware 820 includes multiple qubits 830 arranged in a particular layout. Each qubit of the qubits 830 (including the target and ancilla qubits described herein) can be coupled to an environment and/or to one another. Such coupling(s) decoheres and relaxes quantum information contained in the qubit. Thus, the quantum hardware 820 can be noisy. The type of the multiple links can be based on the type of qubits 830 used by the quantum hardware 820 for computation. In some cases, the multiple links 814 can include wireline links or optical links, or a combination of both. In other cases, the multiple links 814 can include microwave resonator devices or microwave transmission lines, or a combination of both.

The qubits 830 can include atomic qubits assembled in an atom-trap. Thus, the atomic qubits can be referred to as trapped-atom qubits. In some cases, each one of the atomic qubits can be a neutral atom. In other cases, each one of the atomic qubits can be an ion, such as an Ytterbium ion, a calcium ion, or similar ions. The atomic-qubits in such cases can be confined within an ion-trap (e.g., the trap 270 (FIG. 2) and can be assembled in a linear arrangement (such as the linear crystal or chain 110 (FIG. 1)). In other implementations, the qubits 830 can include solid-state devices of one of several types. Such devices can be embodied in, for example, Josephson junction devices, semiconductor quantum-dots, or defects in a semiconductor material (such as vacancies in Si and Ge, or nitrogen-vacancy centers in diamond).

The control subsystem 810 can cause the quantum hardware 820 to execute the quantum circuit and/or sub-circuits as described herein. In response, the control subsystem 810 can receive measurement data 818 indicative of computation outputs that includes the generated samples representing a realization from the modeled joint probability distribution, for example. Because the quantum computation can be performed in two or more qubits, a measurement outcome can be represented as a bitstring representing a particular target output state given a particular set of qubits involved in a quantum computation. The control subsystem 810 can supply at least a portion of the measurement data 818 to components of the control subsystem 810 and/or other subsystems (e.g., post-processing subsystem 850).

The control subsystem 810 also can be functionally coupled to a post-processing subsystem 850 via a communication architecture 840. The communication architecture 840 can include wirelines links, wireless links, network devices (such as gateway devices, servers, and the like), or a combination thereof. The post-processing subsystem 850 can apply one or several post-processing techniques to measurement data 818 received from the quantum hardware 820. By applying such techniques, the post-processing subsystem 850 can generate a result 854 of a quantum computation executed by the quantum hardware. The post-processing subsystem 850 can send the result 854 (or data indicative of the result 854) to the computing device 802 and/or other computing device(s) 858. The post-processing subsystem 850 also can cause the computing device 802 to present the result 854 in a particular way. For example, the post-processing subsystem 850 can direct the computing device 802 to present a user interface including the result 854.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

In general, it is noted that the foregoing description of the disclosure is provided to enable a person skilled in the art to make or use the disclosure. Various modifications to the disclosure will be readily apparent to those skilled in the art, and the common principles defined herein may be applied to other variations without departing from the scope of the disclosure. Furthermore, although elements of the described aspects may be described or claimed in the singular, the plural is contemplated unless limitation to the singular is explicitly stated. Additionally, all or a portion of any aspect may be utilized with all or a portion of any other aspect, unless stated otherwise. Thus, the disclosure is not to be limited to the examples and designs described herein but is to be accorded the widest scope consistent with the principles and novel features disclosed herein.

What is claimed is:

1. A method for optimizing an implementation of a quantum chemistry task in a quantum circuit, comprising:
obtaining a chemistry task presented as a Hamilton to implement in a Variational Quantum Eigensolver (VQE) circuit;
mapping the chemistry task to a qubit space of the VQE circuit with Unitary Coupled Cluster with Single and Double excitations (UCCSD) ansatz comprising a state preparation, single and double fermionic excitations, and a measurement basis, wherein fermionic excitation operators in the qubit space are implemented as Givens rotations dressed with controlled-Z (CZ) gate ladders at a beginning and end of the VQE circuit;
decomposing the fermionic excitations to Givens rotations and CZ gates by passing exponentiated Pauli forms in the VQE circuit to a cloud compiler;
after commuting the CZ gates through Givens rotations, cancelling the commuted CZ gates; and
dropping CZ gates at the beginning and the end of the VQE circuit.

2. The method of claim 1, further comprising executing the VQE having the dropped CZ gates for reducing gate counts in the VQE circuit.

3. The method of claim 1, wherein the state preparation comprises a 1q-qubit X gates.

4. The method of claim 1, further comprising decomposing remaining Givens rotations with two 2q gates for single excitations and thirteen 2q gates for double excitations.

5. The method of claim 4, further comprising alternating polarity of decomposition to cancel an additional 2q gates comprising neighboring 4q Givens rotations.

6. A system for optimizing an implementation of a quantum chemistry task in a quantum circuit, comprising:
at least one processor; and
a memory including instructions that, when executed by the at least one processor, cause the system to:
obtain a chemistry task presented as a Hamilton to implement in a Variational Quantum Eigensolver (VQE) circuit;
map the chemistry task to a qubit space of the VQE circuit with Unitary Coupled Cluster with Single and Double excitations (UCCSD) ansatz comprising a state preparation, single and double fermionic excitations, and a measurement basis, wherein fermionic excitation operators in the qubit space are implemented as Givens rotations dressed with controlled-Z (CZ) gate ladders at a beginning and end of the VQE;
decompose the fermionic excitations to Givens rotations and controlled-Z (CZ) gates by passing exponentiated Pauli forms in the VQE circuit to a cloud compiler;
after commuting the CZ gates through Givens rotations, cancel the commuted CZ gates; and
drop CZ gates at the beginning and the end of the VQE.

7. The system of claim 6, wherein the memory including instructions that, when executed by the at least one processor, further cause the system to execute the VQE having the dropped CZ gates for reducing gate counts in the VQE circuit.

8. The system of claim 6, wherein the state preparation comprises a 1q-qubit X gates.

9. The system of claim 6, wherein the memory including instructions that, when executed by the at least one processor, further cause the system to decompose remaining Givens rotations with two 2q gates for single excitations and thirteen 2q gates for double excitations.

10. The system of claim 9, wherein the memory including instructions that, when executed by the at least one processor, further cause the system to alternate polarity of decomposition to cancel an additional 2q gates comprising neighboring 4q Givens rotations.
